(19) United States
(12) Patent Application Publication
(10) Pub. No.: US 2022/0202650 A1
(43) Pub. Date: Jun. 30, 2022

(54) MEDICINE-TAKING DETECTION SYSTEM AND INGESTION DEVICE

(71) Applicant: Murata Manufacturing Co., Ltd., Nagaokakyo-shi (JP)

(72) Inventors: Kosuke HAMADA, Nagaokakyo-shi (JP); Takanori NAKAMURA, Nagaokakyo-shi (JP); Takaaki ASADA, Nagaokakyo-shi (JP); Risa CHIBA, Nagaokakyo-shi (JP)

(21) Appl. No.: 17/655,058

(22) Filed: Mar. 16, 2022

Related U.S. Application Data
(63) Continuation of application No. PCT/JP2020/036269, filed on Sep. 25, 2020.

(30) Foreign Application Priority Data
Nov. 7, 2019 (JP) 2019-202430

(51) Int. Cl. A61J 7/00; G01S 15/04; A61B 8/08; A61B 8/12

(52) U.S. Cl. CPC A61J 7/0015; G01S 15/04; A61J 2200/30; A61B 8/12; A61J 2200/70; A61B 8/0833

(57) ABSTRACT

A medicine-taking detection system is provided that includes an ingestion device 1 and a detection device that detects taking of the ingestion device. The ingestion device includes a medicine, a signal transmission unit that transmits a first signal, a signal reception unit that receives a reflected signal of the first signal transmitted from the signal transmission unit, and a secondary battery that supplies power to the signal transmission unit and the signal reception unit. The signal transmission unit is configured to, when the signal reception unit receives the reflected signal, transmit a second signal including information on a signal intensity of the reflected signal, and the detection device receives the second signal and detects the taking of the ingestion device based on the information on the signal intensity of the reflected signal.

### CROSS REFERENCE TO RELATED APPLICATIONS

The present application is a continuation of PCT/JP2020/036269 filed Sep. 25, 2020, which claims priority to Japanese Patent Application No. 2019-202430, filed Nov. 7, 2019, the entire contents of each of which are incorporated herein by reference.

### TECHNICAL FIELD

The present invention relates to a medicine-taking detection system and an ingestion device that detect that a medicine is taken.

### BACKGROUND

There is a need for a system that can confirm whether or not a patient takes a prescribed medicine.

An example of such a system is disclosed in Japanese Patent No. 5730822 (hereinafter “Patent Document 1”), which describes a system that detects that, when a medicine device that contains a medicine is taken. In this example, the medicine device is taken by causing a current to flow to a drive circuit included in the medicine device with a conductive liquid such as gastric juice interposed therebetween to transmit a signal and receiving the signal by an external reception device.

However, in the system described in Patent Document 1, though the medicine device is taken, when the medicine device does not react with the conductive liquid, the current does not flow through the drive circuit, and the signal is not transmitted to the outside. In this case, it is not possible to detect that the medicine device is taken.

### SUMMARY OF THE INVENTION

Accordingly, it is an object of the present invention to provide a medicine-taking detection system and an ingestion device configured for accurately detecting that a medicine is taken.

In an exemplary aspect, a medicine-taking detection system includes an ingestion device, and a detection device that detects taking of the ingestion device. The ingestion device includes a medicine, a signal transmission unit that transmits a first signal, a signal reception unit that receives a reflected signal of the first signal transmitted from the signal transmission unit, and a secondary battery that supplies power to the signal transmission unit and the signal reception unit. Moreover, the signal transmission unit is configured to, when the signal reception unit receives the reflected signal, transmit a second signal including information on a signal intensity of the reflected signal, and the detection device receives the second signal, and detects the taking of the ingestion device based on the information on the signal intensity of the reflected signal.

In another exemplary aspect, an ingestion device of the present invention includes a medicine, a signal transmission unit that transmits a first signal, a signal reception unit that receives a reflected signal of the first signal transmitted from the signal transmission unit, a detection unit that detects taking of the medicine based on a signal intensity of the reflected signal, and a secondary battery that supplies power to the signal transmission unit, the signal reception unit, and the detection unit. In this aspect, the signal transmission unit is configured to, when the detection unit detects the taking of the medicine, transmit a second signal indicating that the medicine is taken to an outside.

According to the exemplary medicine-taking detection system of the present invention, since the ingestion device transmits the first signal and receives the reflected signal and detects the taking of the ingestion device based on the signal intensity of the received reflected signal, the taking of the medicine included in the ingestion device is accurately detected.

According to the ingestion device of the present invention, since the ingestion device transmits the first signal, receives the reflected signal, and detects the taking of the medicine based on the signal intensity of the received reflected signal, the ingestion device is configured to accurately detect that the medicine is taken.

### BRIEF DESCRIPTION OF DRAWINGS

FIG. 1 is a block diagram schematically illustrating a configuration of a medicine-taking detection system according to a first exemplary embodiment.

FIG. 2 is a diagram illustrating an example of a change in signal intensity of a reflected signal received by a signal reception unit when an ingestion device is extracted from a state of being stored in a storage member and taken.

FIG. 3 is a diagram illustrating an example of the change in the signal intensity of the reflected signal when the ingestion device is lost without being taken from the storage member after being extracted from the storage member.

FIG. 4 is a block diagram schematically illustrating a configuration of a medicine-taking detection system according to a second exemplary embodiment.

### DETAILED DESCRIPTION

Hereinafter, exemplary embodiments of the present invention will be described, and features of the present invention will be specifically described.

### First Exemplary Embodiment

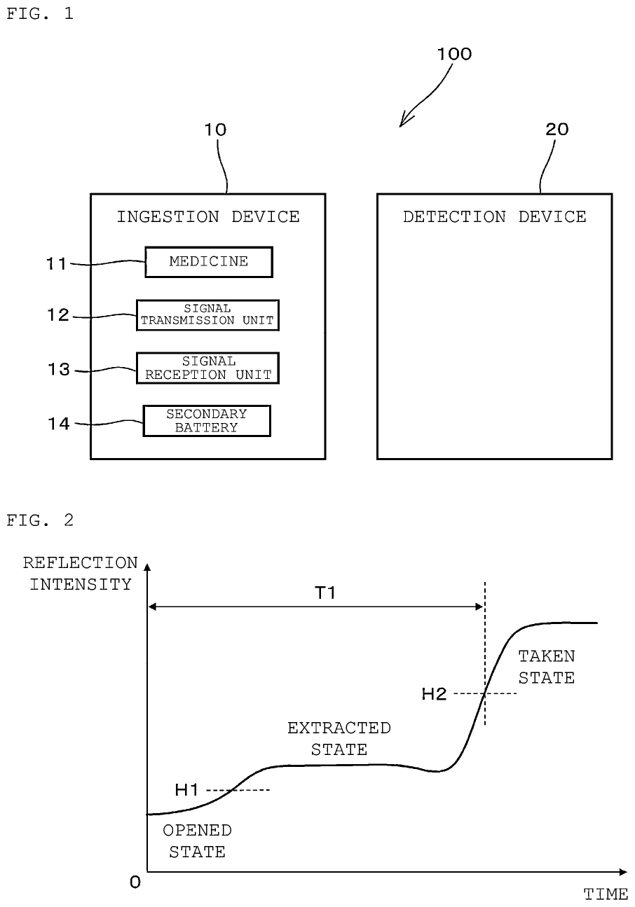

FIG. 1 is a block diagram schematically illustrating a configuration of a medicine-taking detection system 100 according to a first exemplary embodiment. As shown, the medicine-taking detection system 100 according to the first embodiment includes an ingestion device 10 and a detection device 20.

The ingestion device 10 includes a medicine 11, a signal transmission unit 12, a signal reception unit 13, and a secondary battery 14. In exemplary aspects, the shape of the ingestion device 10 is not particularly limited, but has, for example, a capsule-like shape that can be stored in a pocket of a storage member, such as a press through pack sheet (PTP). In this case, the medicine 11, the signal transmission unit 12, the signal reception unit 13, and the secondary battery 14 can be covered with resin. Moreover, the medicine 11 can be included in a manner attached to a surface of the ingestion device 10 or can be included inside the ingestion device 10.

The secondary battery 14 supplies power to the signal transmission unit 12 and the signal reception unit 13. For example, it is preferable that an all-solid-state battery having a solid electrolyte is used as the secondary battery 14. In this case, for example, a solid electrolyte containing a NASICON-type crystal oxide can be used as the solid electrolyte. Since an electrolytic solution does not flow into the body by using an all-solid-state battery not containing an electrolytic solution as the secondary battery 14, safety is high. However, as long as safety can be secured, a battery having an electrolytic solution can be used as the secondary battery 14.

In operation, the signal transmission unit 12 is configured to transmit a first signal. For example, the signal transmission unit 12 is a piezoelectric element, and the first signal is an ultrasonic signal that is a vibration signal. In this case, the ingestion device 10 includes an IC for controlling the piezoelectric element.

Moreover, the signal reception unit 13 is configured to receive a reflected signal of the first signal transmitted from the signal transmission unit 12.

FIG. 2 is a diagram illustrating an example of a change in a signal intensity of the reflected signal received by the signal reception unit 13 when the ingestion device 10 is extracted from a state of being stored in the storage member and taken. Here, a description will be given assuming that the storage member that stores the ingestion device 10 is a PTP sheet.

In a state where the ingestion device 10 is stored in the pocket of the PTP sheet, when the PTP sheet is opened to extract the ingestion device 10, the signal transmission unit 12 transmits the first signal. For example, in a state where the ingestion device 10 is stored in the PTP sheet, charging is performed while a current is continuously supplied to the secondary battery 14. As an example, charging may be performed by providing a current supply line in the PTP sheet and supplying a current to the secondary battery 14 with the current supply line interposed therebetween. When the PTP sheet is opened to extract the ingestion device 10 from this state, the supply of the current to the secondary battery 14 is stopped, and the voltage of the secondary battery 14 decreases. The signal transmission unit 12 can be configured to transmit the first signal by using the decrease in the voltage of the secondary battery 14 as a trigger. In this case, the IC can be configured to detect the decrease in the voltage of the secondary battery 14 described above, and the signal transmission unit 12 can perform control such that the first signal is transmitted. It is noted that the method of detecting that the PTP sheet is opened to extract the ingestion device 10 is not limited to the above-described method.

In a state where the ingestion device 10 is not in contact with a human hand only by opening the PTP sheet, the signal intensity of the reflected signal received by the signal reception unit 13 is substantially constant. This state is referred to as an “opened state” for purposes of this disclosure.

When the ingestion device 10 is extracted from the PTP sheet by the human hand from the opened state, the signal intensity of the reflected signal received by the signal reception unit 13 increases as illustrated in FIG. 2. Here, a state in which the ingestion device 10 is extracted from the PTP sheet and is in contact with the human hand is referred to as an “extracted state” for purposes of this disclosure.

When the ingestion device 10 is taken from the extracted state, since the ingestion device 10 enters the body, as illustrated in FIG. 2, the signal intensity of the reflected signal received by the signal reception unit 13 further increases. Here, a state in which the ingestion device 10 is taken is referred to as a “taken state” for purposes of this disclosure.

When the signal reception unit 13 is configured to receive the reflected signal, the signal transmission unit 12 transmits a second signal including information on the signal intensity of the reflected signal. The information on the signal intensity of the reflected signal can be information on the signal intensity itself or information related to the signal intensity.

Similarly to the first signal, the second signal is, for example, an ultrasonic signal. However, when the first signal and the second signal are the ultrasonic signals, it is preferable that at least one of a frequency and an amplitude of the second signal is different so as to be distinguishable from the first signal. Moreover, the second signal can be transmitted from the signal transmission unit 12 in an encrypted state according to an exemplary aspect.

Moreover, the detection device 20 receives the second signal transmitted from the signal transmission unit 12, and detects the taking of the ingestion device 10 based on the information on the signal intensity of the reflected signal included in the second signal. When the signal transmission unit 12 is the piezoelectric element, for example, the detection device 20 is attached to the body and receives the ultrasonic signal output from the piezoelectric element.

The detection device 20 determines the above-described “opened state”, “extracted state”, and “taken state” based on the magnitude of the signal intensity of the reflected signal. For example, when the signal intensity of the reflected signal becomes larger than a first threshold value H1 (see FIG. 2), the detection device 20 transitions from the “opened state” to the “extracted state”, and determines that the ingestion device 10 is extracted from the storage member. When the signal intensity of the reflected signal becomes larger than a second threshold value H2 (see FIG. 2), the detection device 20 transitions from the “extracted state” to the “taken state”, and determines that the ingestion device 10 is taken. As illustrated in FIG. 2, the second threshold value H2 is larger than the first threshold value H1.

Here, the second signal may include information such as an ID and a lot number of the medicine 10, for example1. In this case, the detection device 20 can also detect a kind and the like of the taken medicine 11. When the second signal is the ultrasonic signal, the detection device may be configured to control a frequency and an amplitude of the vibration of the piezoelectric element according to the ID and the lot number of the medicine 11, and the like.

When the taking of the ingestion device 10 is detected, the detection device 20 can be configured to transmit a notification of the device-taking to a management server (not illustrated). The transmission of the signal to the management server can be performed, for example, by wireless communication.

As described above, in the medicine-taking detection system 100 according to the present embodiment, the signal transmission unit 12 of the ingestion device 10 transmits the first signal, and the signal reception unit 13 receives the reflected signal. The signal transmission unit 12 transmits the second signal including the information on the signal intensity of the reflected signal, and the detection device 20 detects the taking of the ingestion device 10 based on the information on the signal intensity of the reflected signal. As described above, since the signal intensity of the reflected signal is different in each state of the “opened state”, the “extracted state”, and the “taken state”, it is possible to accurately detect that the ingestion device 10 containing the medicine 11 is taken.

In the medicine-taking detection system 100 according to the present embodiment, the detection device 20 can also detect that the ingestion device 10 is extracted from the storage member based on the information on the signal intensity of the reflected signal. That is, it is possible to detect two-stage changes of the transition from the “opened state” to the “extracted state” and the transition from the “extracted state” to the “taken state”, and thus, it is possible to accurately detect that the ingestion device 10 containing the medicine 11 is taken.

Moreover, in an exemplary aspect, the detection device 20 can be configured to record a time at which the ingestion device transitions from “extracted state” to the “taken state”, that is, a time at which the taking of the ingestion device 10 is detected. It is possible to manage not only whether or not the medicine 11 is taken, but also a time when the medicine is taken by recording the taking time of the ingestion device 10.

The detection device 20 can also be configured to detect that the ingestion device 10 is lost without being taken after being extracted from the storage member based on the information on the signal intensity of the reflected signal included in the second signal transmitted from the signal transmission unit 12.

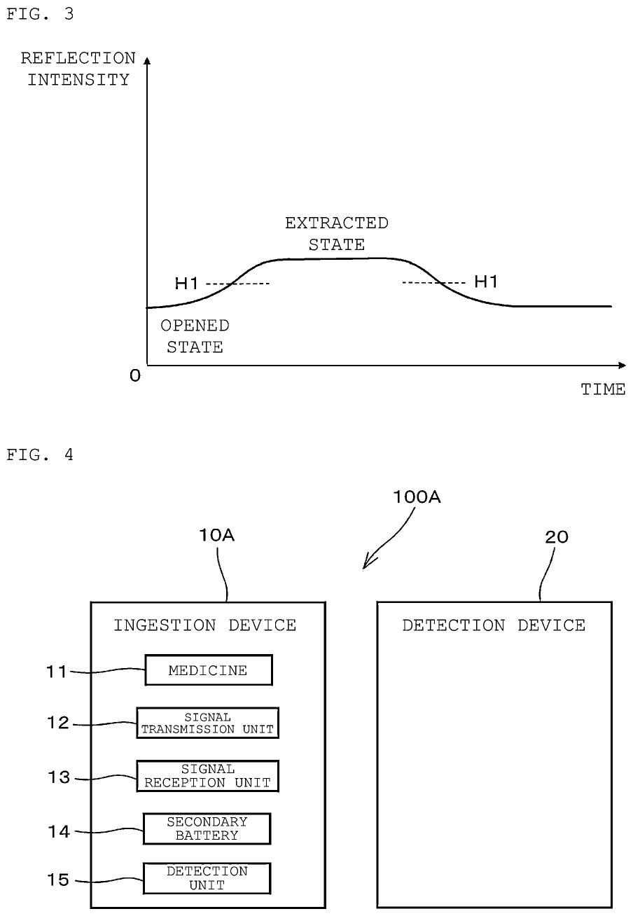

FIG. 3 is a diagram illustrating an example of a change in the signal intensity of the reflected signal when the ingestion device 10 is lost without being taken after being extracted from the storage member. As illustrated in FIG. 3, the change in the signal intensity of the reflected signal when the ingestion device 10 is extracted from the storage member and transitions from the opened state to the extracted state is the same as that illustrated in FIG. 2.

When a user loses the ingestion device 10 for reasons such as dropping the ingestion device after taking the ingestion device out of the storage member, the signal intensity of the reflected signal decreases to a magnitude equal to or lower than the “opened state”, and this state is continued. The detection device 20 detects that the signal intensity of the reflected signal decreases below the first threshold value H1 from the “extracted state”, or determines that the ingestion device 10 is lost without being taken when the signal intensity of the reflected signal decreases below the first threshold value H1 and this state is continued for a predetermined time or more. When the loss of the ingestion device 10 is detected, the detection device 20 can be configured to transmit a notification of the loss to the management server (not illustrated).

When the user loses the ingestion device 10, the user may extract the ingestion device 10 of the same kind from the storage member and takes the ingestion device. The detection device 20 can then be configured to detect the decrease in the signal intensity of the reflected signal described above, and can also determine that the ingestion device 10 in which the signal intensity of the reflected signal decreases is lost when the taking of another ingestion device 10 is detected. According to this method, it is possible to accurately detect the loss of the ingestion device 10.

The detection device 20 can also be configured to measure a time from when the ingestion device 10 is extracted from the storage member to when the ingestion device is taken based on the information on the signal intensity of the reflected signal included in the second signal. In this case, the detection device 20 measures a time T1 (see FIG. 2) from when the second signal transmitted from the signal transmission unit 12 is received to when the transition of the ingestion device to the taken state is detected, and obtains the measured time T1 as a time from when the ingestion device 10 is extracted from the storage member to when the ingestion device is taken. The measured time from when the ingestion device 10 is extracted from the storage member to when the ingestion device is taken can be used, for example, for instruction of a method for taking the medicine.

The detection device 20 can also be configured to determine that an abnormality occurs when it is detected that the signal intensity of the reflected signal is equal to or less than the second threshold value H2 or is equal to or less than the first threshold value H1 after the signal intensity exceeds the second threshold value H2.

In an exemplary aspect, the detection device 20 can also include a sensor for detecting vital signs such as body temperature and pulse. For example, it is possible to grasp the influence of the medicine 11 on the body by detecting vital signs before and after the ingestion device 10 is taken.

### Second Exemplary Embodiment

In the medicine-taking detection system 100 according to the first embodiment, the detection device 20 is configured to receive the second signal transmitted from the ingestion device 10, and detect the taking of the ingestion device 10 based on the information on the signal intensity of the reflected signal included in the second signal.

On the other hand, in a medicine-taking detection system 100A according to a second exemplary embodiment, the ingestion device 10 itself detects that the medicine is taken, and transmits a second signal indicating that the medicine is taken to the outside.

FIG. 4 is a block diagram schematically illustrating a configuration of the medicine-taking detection system 100A according to the second exemplary embodiment. In FIG. 4, the same components as those of the medicine-taking detection system 100 illustrated in FIG. 1 are denoted by the same reference symbols, and a detailed description thereof is omitted.

As shown, the medicine-taking detection system 100A according to the second embodiment includes an ingestion device 10A and a detection device 20.

The ingestion device 10A includes a medicine 11, a signal transmission unit 12, a signal reception unit 13, a secondary battery 14, and a detection unit 15.

Similarly to the medicine-taking detection system 100 according to the first embodiment described above, the signal transmission unit 12 is configured to transmit a first signal, and the signal reception unit 13 is configured to receive a reflected signal of the first signal transmitted from the signal transmission unit 12. Moreover, the secondary battery 14 supplies power to the signal transmission unit 12, the signal reception unit 13, and the detection unit 15.

The detection unit 15 includes, for example, an IC, and detects the taking of the medicine 11 based on a signal intensity of the reflected signal received by the signal reception unit 13. Similarly to the detection device 20 of the medicine-taking detection system 100 according to the first embodiment, the detection unit 15 determines an “opened state”, an “extracted state”, and a “taken state” based on a magnitude of the signal intensity of the reflected signal. For example, when the signal intensity of the reflected signal becomes larger than a first threshold value H1, the detection unit 15 transitions from the “opened state” to the “extracted state”, and determines that the ingestion device 10 is extracted from the storage member. When the signal intensity of the reflected signal becomes larger than a second threshold value H2, the detection unit 15 transitions from the “extracted state” to the “taken state”, and determines that the medicine 11 is taken.

When the detection unit 15 detects the taking of the medicine 11, the signal transmission unit 12 is configured to transmit the second signal indicating that the medicine 11 is taken to the outside. The detection device 20 detects the taking of the medicine 11 by receiving the second signal transmitted from the signal transmission unit 12.

Similarly to the medicine-taking detection system 100 according to the first embodiment, in the medicine-taking detection system 100A according to the second embodiment, it is possible to accurately detect that the ingestion device 10 containing the medicine 11 is taken. In particular, in the medicine-taking detection system 100A according to the second embodiment, the ingestion device 10 itself containing the medicine 11 can detect the taking of the medicine 11.

Similarly to the detection device 20 of the medicine-taking detection system 100 according to the first embodiment, the detection unit 15 can also be configured to detect that the ingestion device 10 is lost without being taken after being extracted from the storage member based on the signal intensity of the reflected signal. The detection unit 15 can be configured to measure a time from when the ingestion device 10 is extracted from the storage member to when the ingestion device is taken based on the signal intensity of the reflected signal.

In general, it is noted that the present invention is not limited to the above-described embodiments, and various applications and modifications can be added within the scope of the present invention.

### DESCRIPTION OF REFERENCE SYMBOLS

10, 10A: Ingestion device

11: Medicine

12: Signal transmission unit

13: Signal reception unit

14: Secondary battery

15: Detection unit

20: Detection device

100, 100A: Medicine-taking detection system

1. A medicine-taking detection system comprising:
an ingestion device; and
a detection device configured to detect a taking of the ingestion device and including:
a medicine,
a signal transmission unit configured to transmit a first signal,
a signal reception unit configured to receive a reflected signal of the first signal transmitted from the signal transmission unit, and
a secondary battery configured to supply power to the signal transmission unit and the signal reception unit,
wherein the signal transmission unit is configured to transmit a second signal including information relating to a signal intensity of the reflected signal when the signal reception unit receives the reflected signal, and
wherein the detection device is configured to receive the second signal and to detect the taking of the ingestion device based on the information relating to the signal intensity of the reflected signal.

2. The medicine-taking detection system according to claim 1,
wherein the ingestion device is stored in a storage member, and
wherein the detection device is configured to detect that the ingestion device is extracted from the storage member based on the information relating to the signal intensity of the reflected signal included in the second signal.

3. The medicine-taking detection system according to claim 2, wherein the detection device is configured to detect that the ingestion device is lost without being taken after being extracted from the storage member based on the information relating to the signal intensity of the reflected signal included in the second signal.

4. The medicine-taking detection system according to claim 2, wherein the detection device is configured to measure a time from when the ingestion device is extracted from the storage member to when the ingestion device is taken based on the information relating to the signal intensity of the reflected signal included in the second signal.

5. The medicine-taking detection system according to claim 1, wherein the first signal is an ultrasonic signal.

6. The medicine-taking detection system according to claim 1, wherein the ingestion device comprise a capsule-like shape configured to be stored in a pocket of a storage member.

7. The medicine-taking detection system according to claim 6, wherein the storage member comprises a press through pack sheet (PTP).

8. The medicine-taking detection system according to claim 7, wherein, when the PTP is opened to extract the ingestion device, a supply of power to the secondary battery is stopped, such that a voltage of the secondary battery decreases.

9. The medicine-taking detection system according to claim 8, wherein the signal transmission unit is configured to transmit the first signal by using the decrease in the voltage of the secondary battery as a trigger.

10. The medicine-taking detection system according to claim 1, wherein the signal transmission unit is a piezoelectric element and the ingestion device includes an IC configured to control the piezoelectric element.

11. The medicine-taking detection system according to claim 1, wherein the signal transmission unit is further configured to transmit the second signal to include identification information of the medicine.

12. An ingestion device comprising:
a signal transmission unit configured to transmit a first signal;
a signal reception unit configured to receive a reflected signal of the first signal transmitted from the signal transmission unit;
a detection unit configured to detect a taking of a medicine contained in the ingestion device based on a signal intensity of the reflected signal;
a secondary battery configured to supply power to the signal transmission unit, the signal reception unit, and the detection unit,
wherein the signal transmission unit is configured to transmit a second signal indicating that the medicine is taken outside the ingestion device when the detection unit detects the taking of the medicine.

13. The ingestion device according to claim 12,
wherein the ingestion device is stored in a storage member, and
wherein the detection unit is configured to detect that the ingestion device is extracted from the storage member based on the signal intensity of the reflected signal.

14. The ingestion device according to claim 13, wherein the detection unit is further configured to detect that the ingestion device is lost without being taken after being extracted from the storage member based on the signal intensity of the reflected signal.

15. The ingestion device according to claim 14, wherein the detection unit is configured to measure a time from when the ingestion device is extracted from the storage member to when the ingestion device is taken based on the signal intensity of the reflected signal.

16. The ingestion device according to claim 12, wherein the first signal is an ultrasonic signal.

17. The ingestion device according to claim 13, wherein the storage member comprises a press through pack sheet (PTP).

18. The ingestion device according to claim 17, wherein, when the PTP is opened, a supply of power to the secondary battery is stopped, such that a voltage of the secondary battery decreases.

19. The ingestion device according to claim 18, wherein the signal transmission unit is configured to transmit the first signal by using the decrease in the voltage of the secondary battery as a trigger.

20. The ingestion device according to claim 12, wherein the signal transmission unit is a piezoelectric element and the ingestion device further comprises an IC configured to control the piezoelectric element.
